(19) United States
(12) Patent Application Publication
(10) Pub. No.: US 2003/0078610 A1
(43) Pub. Date: Apr. 24, 2003

(54) Medical cast aerator hole cutting device

(76) Inventor: John J. Yedlowski, Charlotte Hall, MD (US)

Correspondence Address: Richard C. Litman, LITMAN LAW OFFICES, LTD., P. O. Box 15035, Arlington, VA, 22215, US

(21) Appl. No.: 10/226,301

(22) Filed: Aug. 23, 2002

Related U.S. Application Data
(60) Provisional application No. 60/330,554, filed on Oct. 24, 2001.

(51) Int. Cl. A61B 17/14

(52) U.S. Cl. 606/179

(57) ABSTRACT

A drill bit for cutting annular holes in medical casts without cutting into the covered skin surface. The drill bit includes, an elongated one-piece tubular body having a first end portion and a second end portion. The first end portion is closed except for a centered hole used to attach the drill bit to a driver. The second end portion incorporates a plurality of non-offset equilateral triangle shaped saw-like cutting surfaces machined into the circumference of the second end portion. When attached to a driver imparting a reciprocating motion, the drill bit responds with a self aligning bidirectional cut in the cast matrix obviating the need for a pilot bit. As a result, a cast core is safely cut without damage to the covered skin leaving a hole for aeration of the skin surface.

### CROSS REFERENCE TO RELATED APPLICATION

This application claims the benefit of U.S. Provisional Patent Application Serial No. 60/330,554, filed Oct. 24, 2001.

### BACKGROUND OF THE INVENTION

1. Field of the Invention

The present invention relates generally to a drill bit device. More specifically, the invention is a medical cast aerator cutting device used to cut cores safely, primarily from medical casts, to provide indirect aeration of the underlying skin surface through a breathable liner.

2. Description of Related Art

Thousands of people and animals break bones each year. These injuries often require that the broken bones be reset and held in place by a hard surface cast until the broken bone mends. The casts are generally made from a fiberglass or plaster mixture to form a solid surface lacking any form of aeration of the underlying skin. As a result, the skin may become excessively moist due to restricted perspiration causing breakdown of the skin. The invention, which is a hole saw blade bit, solves this problem by allowing circular cores of the cast to be removed from the cast to permit aeration of the skin at desired locations on the cast.

Previous inventions describe two general categories of blades for use on materials other than casts. The first category are blades requiring the use of a pilot bit for causing an initial central cut for alignment and grip of the external unidirectional cutting teeth. The second category of hole saw type blades do not use pilot bits, but are blades structurally designed for specific applications other than cutting through casts, and, thus the teeth are designed accordingly.

In the first category, U.S. Pat. No. 4,422,811, issued on Dec. 27, 1983, to Bruce Ellison et al.; U.S. Pat. No. 3,758, 221, issued on Sept. 11, 1973, to Avram M. Meshulam; and U.S. Pat. No. 1,645,736, issued on Oct. 18, 1927, to Sven M. Blanch et al.; all describe hole saw blades comprising an inner pilot bit or drill for priming the surface with an initial central cut, and allowing for alignment and grip of the external cutting teeth. Once the initial central cut is made, the external cutting teeth come into contact with the desired surface to make a cylindrical cut. Since the above patents require the use of pilot bit, and the drawings in each of the above patents appear to depict non-equilateral shaped teeth or “wolf” teeth with a strong angular bias to one side and offset, these hole saw bits are designed for a unidirectional and unrefined cut with the strong possibility of the pilot bit puncturing the patient if the blade were to be tried for cutting cores in casts. In addition, none of the above patents describe a hole saw blade with ejection portal slots for removing the cast core upon completion of the cut. The above patents, therefore, do not depict a hole saw blade without a bit or equilateral triangular shaped teeth with cutting surfaces capable of a self-aligning bidirectional cut when attached to an apparatus causing the blade to move in a reciprocating back and forth motion.

Similarly, U.K. Patent Application No. 1 593 357, published on Jul. 15, 1981, for Masaaki Miyanga, describes a hole saw blade for cutting holes in plate glass. Miyanga also describes the use of an inner pilot bit or center pin to avoid eccentricity of the drill, i.e., misalignment of the cylindrical annular blade. Miyanga further describes the use of grinding teeth of hard agglomerated diamond powder or particles at its cutting edges for cutting into the glass surface. These types of teeth in conjunction with the pilot bit suggest that the hole saw blade can only create a unidirectional cut, and would likely create an unrefined gouge in a plaster cast to possibly injure the underlying skin surface. In addition, Miyanga does not describe a hole saw blade with ejection portal slots for removing the cast core upon completion of the cut. Miyanga, therefore, does not describe or depict the use of a hole saw blade without a bit or equilateral triangular shaped teeth capable of a self-aligning bidirectional cut, when attached to an apparatus causing the blade to move in a reciprocating back and forth motion.

In the second category of previous inventions, U.K. Patent Application No. 2 104 808 A, published on Mar. 16, 1983, for Harry P. Muscroft et al., describes a hole cutter to be used with a twist drill to produce a large hole in a thin panel, e.g., sheet metal or duct for an electric cable. Muscroft et al. describes a hole cutter without a pilot bit, but instead contains an axial hole for accommodating a grub screw used to clamp the twist drill in position. This structural arrangement functions similarly or equivalently to the integrated pilot bit as it uses a centering structure, i.e., the grub screw, for stabilizing and aligning the hole cutter. The Muscroft et al. design further depicts the use of an annular collar to prevent the hole blade from boring too deep into the thin surface, and contains serrated teeth formed with cutting edges not disposed in a plane perpendicular to the axis of the cutter, but at an angle, so that the radially outer edge of each tooth stands axially apart from the remainder of the teeth. As a result, this design functions to provide a very shallow cut, and is explicitly used with a twist drill only capable of non reciprocating motion. Thus, this blade could not be safely used to cut cores into cast material. Muscroft et al., consequently, does not describe a hole saw blade with ejection portal slots for removing a cast core upon completion of a cut. Muscroft et al., therefore, does not describe or depict the use of a hole saw blade with the use of a pilot bit type structure or with equilateral triangular shaped teeth, wherein the base of such teeth are oriented in a plane perpendicular to the axis of the tubular element, and capable of a self-aligning bidirectional cut when attached to an apparatus causing the blade to move in a reciprocating back and forth motion.

Similarly, U.S. Pat. No. 6,267,025 B1, issued on Jul. 31, 2001, to Paul M. Sand et al. describes a tool for extracting broken screws from bone or wood, having an elongated shaft with cutting edges and inner tapered threads engaging the broken screw. This screw extractor is positioned over the screw to be removed, and is rotated in one direction to allow the cutting edges to cut away the surrounding bone or wood until the inner tapered threads bite into the screw so that it may be rotated out of the substrate. Clearly, this device, although useful, is designed to be rotated in a non-reciprocating motion while cutting the surrounding material, i.e., bone or wood, in order to access and grip the broken screw for removal. This device is not designed to cut cores from casts, particularly, as the broken screw in effect acts as a pilot bit in an opposite direction as the broken screw is extracted from the substrate. Sand et al., consequently, does not describe a hole saw blade with ejection portal slots for removing a cast core upon completion of a cut. Sand et al., therefore, does not describe or depict the use of a hole saw blade without the use of a pilot bit type structure or with equilateral triangular shaped teeth, wherein the base of such teeth are oriented in a plane perpendicular to the axis of the tubular element capable of a self-aligning bidirectional cut when attached to an apparatus causing the blade to move in a reciprocating back and forth motion.

U.S. Pat. No. 5,996,571, issued on Dec. 7, 1999, to Andrew L. Jedick, is another example of a drill bit designed for a specific function other than cast cutting. Jedick describes a diamond core drill bit for cutting annular holes in construction materials such as concrete, asphalt, masonry, rock, and stone. As described, this bit is attached to a motorized drill assembly, and rotated in a single direction to cut a core in the construction material. Jedick does not describe the use of a pilot bit, but uses a cutting head composed of a plurality of cutting segments. Each cutting segment, which is depicted as a rectangular and not an equilateral triangle shape, is composed of an outer, middle and inner portion with an uneven dispersion of diamond particles. This dispersion pattern apparently provides better tracking and cutting compared to conventional bits of uniform diamond dispersion. Since these rectangular diamond blades are used for making a unidirectional, i.e., non-reciprocating, cut in construction material, this blade could not be safely used to cut a core in much softer cast material through the use of a more gentle bidirectional reciprocating motion. Jedick, consequently, does not describe a hole saw blade with ejection portal slots for removing a cast core upon completion of a cut. Jedick, therefore, does not describe or depict the use of a hole saw blade with equilateral triangular shaped teeth, wherein the base of such teeth are oriented in a plane perpendicular to the axis of the tubular element, and are capable of a self-aligning bidirectional cut when attached to an apparatus causing the blade to move in a reciprocating a back and forth motion.

U.S. Pat. No. 6,223,637 B1, issued on May 1, 2001, to Peter T. Hansen, describes a hole cutter adapted for cutting holes through the sidewalls of medical catheters. Hansen describes a hollow tube having a distal cutting end with a lumen of smaller diameter near the cutting end, wherein slugs formed at the cutting end are extracted by suction applied through the hollow tube. Upon application of a device to impart motion, the cutting edge is rotated in one direction as indicated by the arrow 3, in FIG. 1, forming a radial hole approximately 0.01 inch to approximately ⅜ inch, with the very small slug removed by suction. Clearly, this structure is designed to function on very small catheters possibly inside a human being, and cannot be scaled or adopted for use in cutting cores out of cast material. Hansen does not describe a hole saw blade with ejection portal slots for removing a cast core upon completion of a cut. Hansen does not describe or depict the use of a hole saw blade with equilateral triangular shaped teeth, wherein the base of such teeth are oriented in a plane perpendicular to the axis of the tubular element capable of a self-aligning bidirectional cut when attached to an apparatus causing the blade to move in a reciprocating back and forth motion.

Similarly, U.S. Pat. No. 6,042,581, issued on Mar. 28, 2000, to Timothy J. Ryan et al. describes a catheter assembly for performing transmyocardial revascularization through the coronary arteries or veins. This assembly depicts a boring catheter tip with radial cutting blades with cutting edges facing the single direction of rotation indicated by arrow 64 in FIG. 10. Basically, upon placement through the coronary blood vessels to the heart, the device is rotated, whereby the cutting edges form a small hole or channel through the myocardial tissue, and the tissue removed via the catheter. Specifically, the boring tip may have a sharp or serrated edge of 0.1 inch diameter with a blade pitch of approximately 45° in order to drive the cut myocardial tissue into the lumen of the coring catheter. Similar to the Hansen invention, the Ryan et al. invention is structurally designed to function inside very small human structures and tissue, and cannot be scaled or adopted for use in cutting cores out of cast material. Ryan et al., further, does not describe a hole saw blade with ejection portal slots for removing a cast core upon completion of a cut. Ryan et al., therefore, does not describe or depict the use of a hole saw blade with equilateral triangular shaped teeth, where such teeth are oriented in a plane perpendicular to the axis of the tubular element, and capable of a self-aligning bidirectional cut when attached to an apparatus causing the blade to move in a reciprocating back and forth motion.

Finally, U.S. Pat. No. 5,468,247, issued on Nov. 21, 1995, to John T. Matthai et al. describes an oscillating driveable chuck and a blade affixed to the chuck with a safety lock means which permits the blade to cut horizontally across a cast or a bone when driven by a conventional high speed, low amplitude oscillating saw handpiece. The structure is consistent with the function, as the driveable chuck and blade is essentially an elliptical shaped flat platform with cutting edges pointing radially outward from the attachment shaft of the conventional oscillating powered medical handpiece, and specifically designed to cut a cast or bone. The Matthai et al. structure, further, does not have cutting surfaces in the shape of equilateral triangles with the base of the triangles oriented in a plane perpendicular to the axis of the tubular element, and the top of the cutting surfaces pointing downward in a direction parallel to the length of the tubular element. Matthai et al., specifically, does not describe an elongated tubular hole saw blade for receiving a cast core and an ejection portal slot for removing a cast core upon completion of a cut. Matthai et al., further, does not describe or depict the use of an elongated tubular hole saw blade with equilateral triangular shaped teeth, wherein the base of such teeth are oriented in a plane perpendicular to the axis of the tubular element, and capable of a self-aligning bidirectional cut when attached to an apparatus causing the blade to move in a reciprocating motion. The Matthai et al. saw blade, therefore, cannot be used to cut cores due to the significant structural and functional differences from the applicant's medical cast aerator device.

None of the above inventions and patents, taken either singly or in combination, is seen to describe the instant invention as claimed.

### SUMMARY OF THE INVENTION

The present invention is directed to a unique drill bit device for cutting annular holes in medical casts without cutting the covered skin surface. The drill bit device includes a cylindrical body having a first end portion and a second end portion. The first end portion is closed except for a centered hole used to attach the drill bit device to a driver tool. At least one and preferably two opposed portal slots are located on the side of the body for enabling the removal of the cut plug by another tool. The slots can have different shapes such as an oblong or a truncated oblong. The second end portion incorporates a plurality of non-offset equilateral triangle shaped saw-like cutting surfaces machined into the circumference of the second end portion. The base and the tops of the cutting edges of the triangular cutting surfaces are parallel and perpendicular to the axis of the cylindrical body. When attached to a driver machine imparting a reciprocating motion, the drill bit device responds with a self aligning bidirectional cut in the cast matrix, obviating the need for a pilot bit. As a result, a cast core is safely cut without damage to the patient's skin surface leaving a hole for aeration of the skin surface through a liner.

Accordingly, it is a principal object of the invention to provide a safe medical cast aerator device for cutting and removing plugs or cores from a cast on a patient to provide skin aeration through the liner, as opposed to a straight line horizontal cut along the length of a cast for removal of the cores or plugs.

It is another object of the invention to provide a medical cast aerator device capable of transferring a reciprocating motion into a bidirectional cut in a cast without the use of a pilot bit.

It is a further object of the invention to provide a medical cast aerator device having at least one keyway for facilitating the removal of the cut cast core or plug from the device.

Still another object of the invention is to securely mount the medical cast aerator device on a mandrel or stud of a rotary reciprocating drill.

It is an object of the invention to provide improved elements and arrangements thereof in an apparatus for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1A is an environmental, perspective view of a medical cast aerator device after forming an aerating hole in a cast on an appendage according to the present invention.

FIG. 1B is an environmental perspective view of a cast on an appendage with an aerating core removed.

FIG. 2 is a cross-sectional view of the medical cast aerator cutter device showing a cylindrical wall.

FIG. 3 is a top plan view of a first end portion and its oscillating/reciprocating motion in directional arrowheads.

FIG. 4 is an environmental, perspective of a cast cutter machine in shadow and the attached medical cast aerator cutter device.

FIG. 5A is a perspective side view of a first embodiment of a medical cast aerator cutter device with a hexagonal attachment hole.

FIG. 5B is a perspective side view of a second embodiment of a medical cast aerator cutter device with a circular attachment hole.

FIG. 6 is an enlarged scale, partially exploded, perspective view of an assembly of mandrel attachments in the medical cast aerator cutter device, partly in section.

FIG. 7 is a bottom plan view of the second end portion of a medical cast aerator cutter device.

FIG. 8A is an enlarged, partial, side elevational view of a cutting surface or teeth of the cutter device.

FIG. 8B is an enlarged bottom plan view of a portion of the cutting surface or teeth of the cutter device.

FIG. 9 is a plan view of one cutting surface of a tooth of the cutter device.

Similar reference characters denote corresponding features consistently throughout the attached drawings.

### DETAILED DESCRIPTION OF THE PREFERRED EMBODIMENT

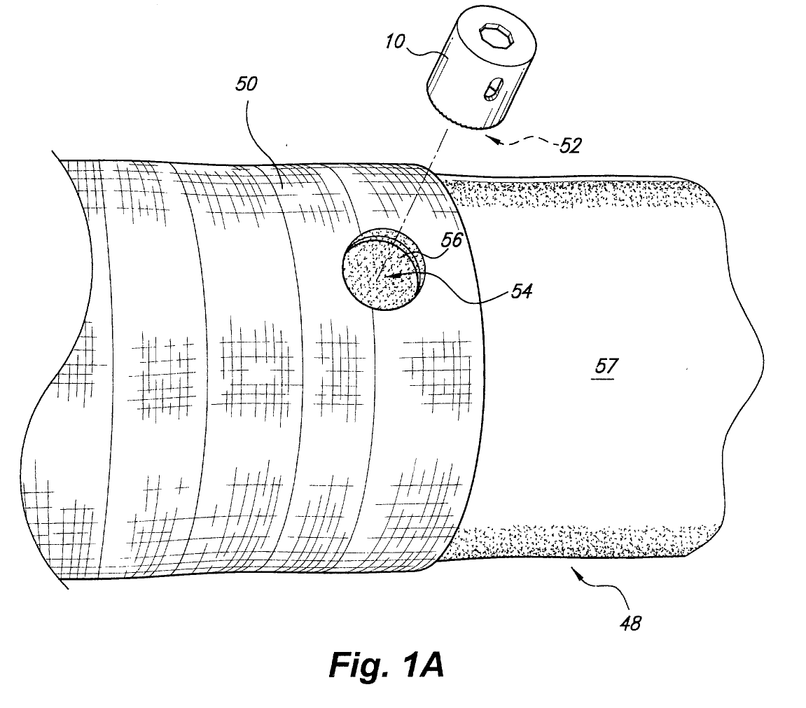

The present invention in FIG. 1A refers to a medical cast aerator cutting device 10 used to cut safely, at least one cylindrical core 52 (hidden) from a medical cast 50 on a body part 48 to provide indirect aeration of the underlying skin 57 of a patient through a cast liner 56.

In FIGS. 1A and 1B, the device 10 has safely cut a cast plug or core 52 from the medical cast 50 leaving a core hole 54 without damaging the underlying liner 56 and the skin 57 of the patient's body part 48. The cylindrical device 10 can have a variety of lengths and diameters to cut cores safely for aerating the skin 57, and in a preferred embodiment, the device 10 is approximately 1.5 inches in length and 1 inch in diameter.

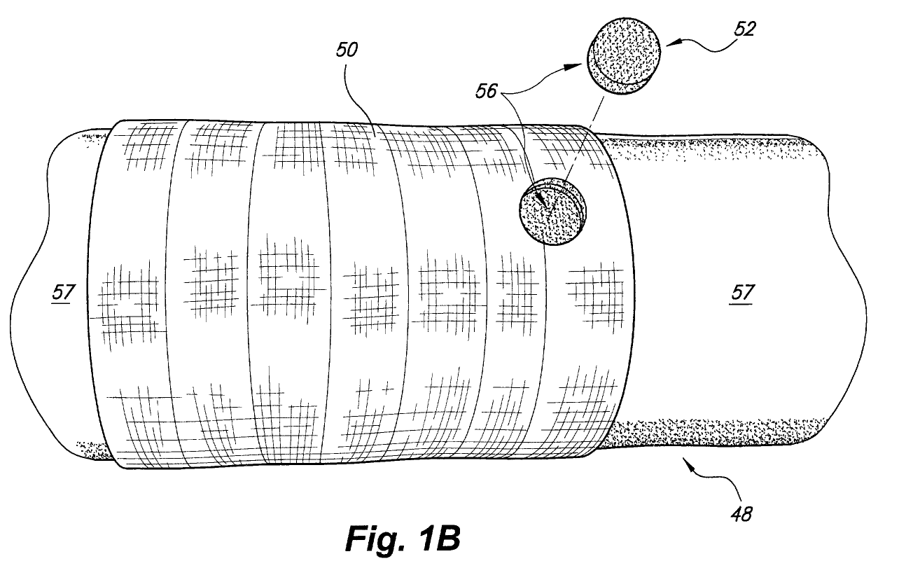

The medical cast aerator cutting device 10, as shown in FIG. 2, includes an integrated one-piece cylindrical body or tube 13 having a first end portion 12 and an opposed second end portion 14. The device 10 is made from a variety of grade metals, metal alloys and metallic compounds, which have the necessary properties to allow exact tempering and finishing. In a preferred embodiment, the device 10 is made from stainless steel.

In FIG. 2, the cylindrical tube 13 has a sidewall thickness that is “back tapered” varying in thickness along the length of the elongated tube, i.e., the second outer end portion 14 has a wall thickness T¹ slightly thicker than the wall thickness T of the first end portion 12. In a preferred embodiment, the wall thickness of the second outer end portion T¹ is approximately 0.002 inch thicker than the wall thickness of the first inner end portion T.

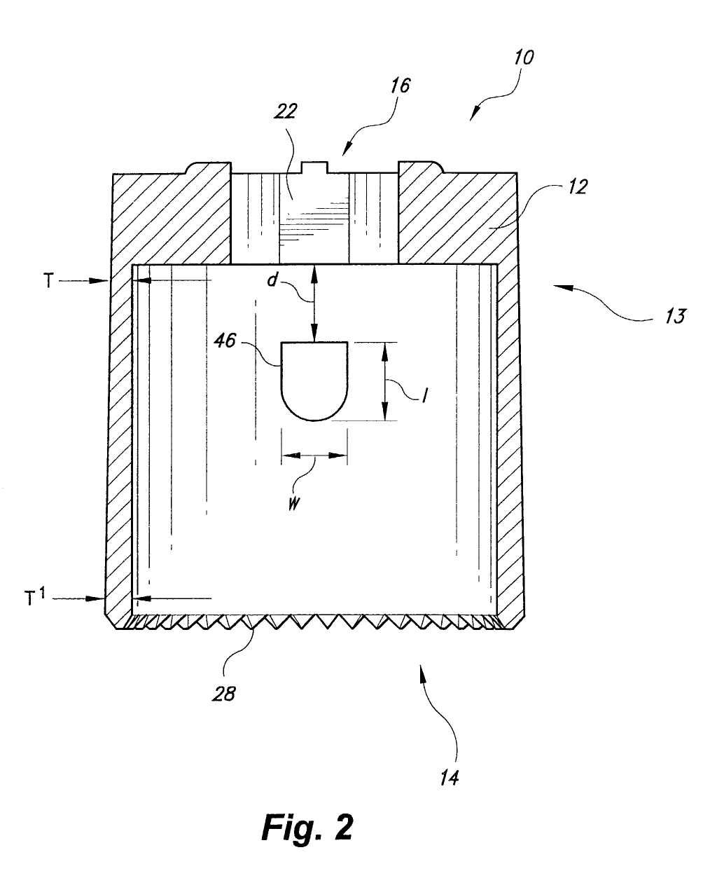

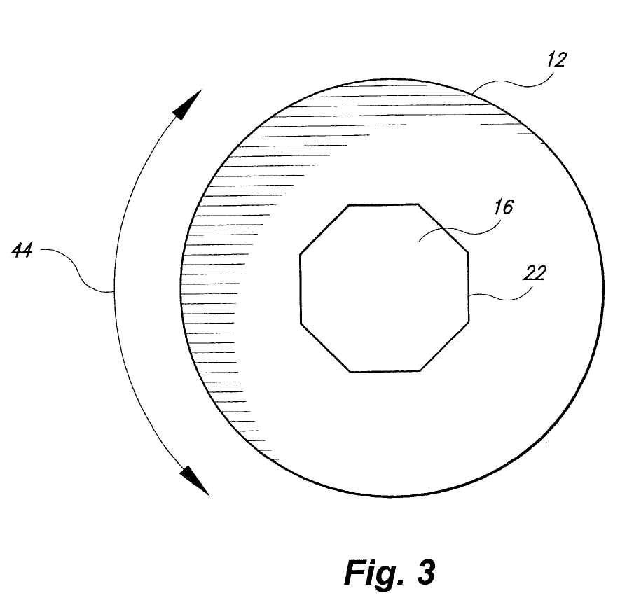

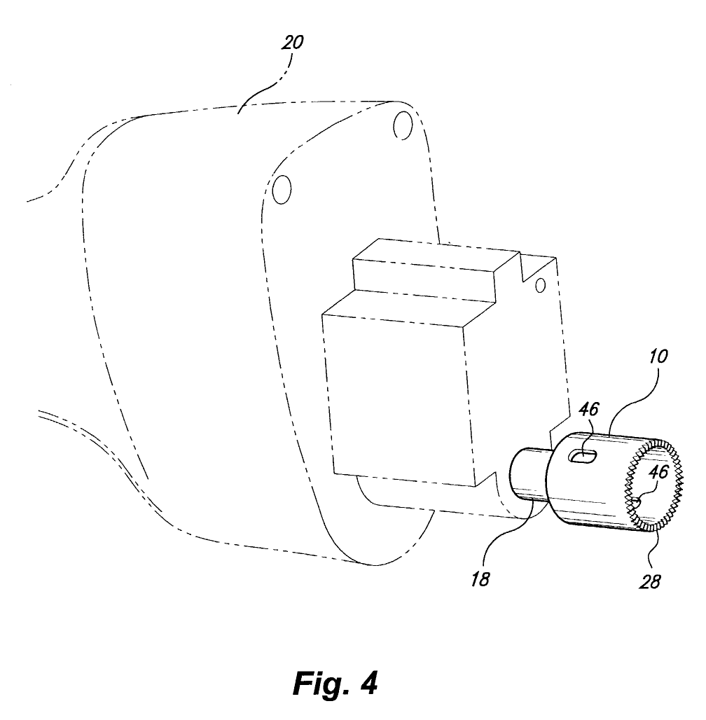

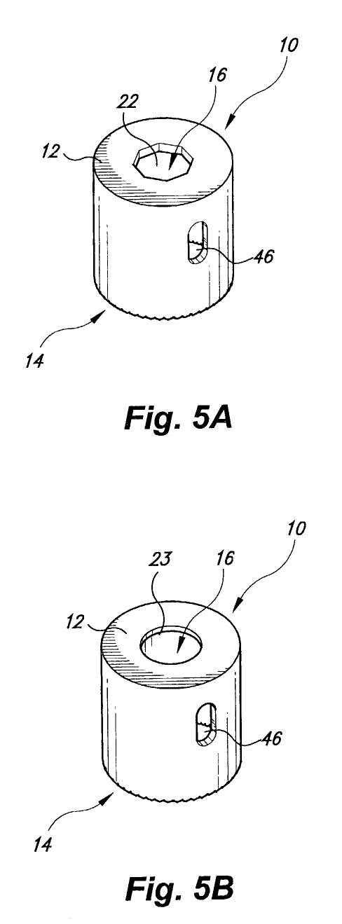

In FIGS. 2 and 3, the first end portion 12 of the device 10 has a center hole 16 having a shape, e.g., octagonal, capable of receiving a correspondingly shaped mandrel or stud portion 18 of a cast cutter machine 20 as depicted in FIG. 4. The first end portion 12 (FIGS. 2 and 3) has an adequate thickness to provide stability of the device 10 on the mandrel 18 (FIG. 3). Further, the center hole 16 can have a variety of circular (23, FIGS. 5B and 6) and non-circular shapes, e.g., hexagonal or octagonal (22 in FIGS. 1A, 2, 3, 5A, and 7), to ensure a secure attachment to the stud or mandrel 18. In a preferred embodiment, the center hole 22 has a maximum diameter of ⅜ inch and an octagonal shape. An alternate embodiment to a non-circular hole is a circular hole 23 having a similar diameter.

Since the second end portion 14 is open ended to receive a core cut 52 from a cast 50, the cylindrical body 13 as in FIG. 2 has at least one truncated portal slot 46, and preferably, two ejection portal slots 46 (FIG. 4). The portal slots 46 are holes located diametrically opposite each other in the elongated tubular body 13, separated by 180°. The ejection portal slots 46 can have a variety of shapes such as the truncated oblong shape in FIG. 2, or oblong shapes illustrated in FIGS. 4, 5A, 5B, and 6. The ejection portal slots 46 are located at least a distance d (FIG. 2) of 0.25 inch from the inside surface 56 of the first end portion 12. Each ejection portal slot 46 is at least ⅜ inch in length 1 (FIG. 2) and at least 5/32 inch in width w. When a cast plug (“core”) 52 as in FIG. 1B is cut with the medical cast aerator cutting device 10 leaving a core hole 54, the cast plug 52 often becomes lodged in the inner portion 58 (FIG. 6) of the cylindrical body 13, as the second end portion 14 is open ended. When a screwdriver or another similar device is inserted into either ejection portal slot 46, the cast plug 52 is manually forced out of the inner portion of the cylindrical body 13. Consequently, the created core hole or holes 54 permit aeration of the underlying liner 56 and skin surface 57. The portal slots 46 may also be used to remove the tool 10 from the mandrel 18 by inserting a screwdriver blade or similar device through the slot 46 to pry the tool 10 off of the mandrel 18.

After the device 10 in FIG. 6 is positioned on the mandrel 18, a lock washer 24 and a bolt 26 are sequentially disposed on the mandrel 18 to secure the device 10 to the mandrel portion 18 of the cast cutter machine 20.

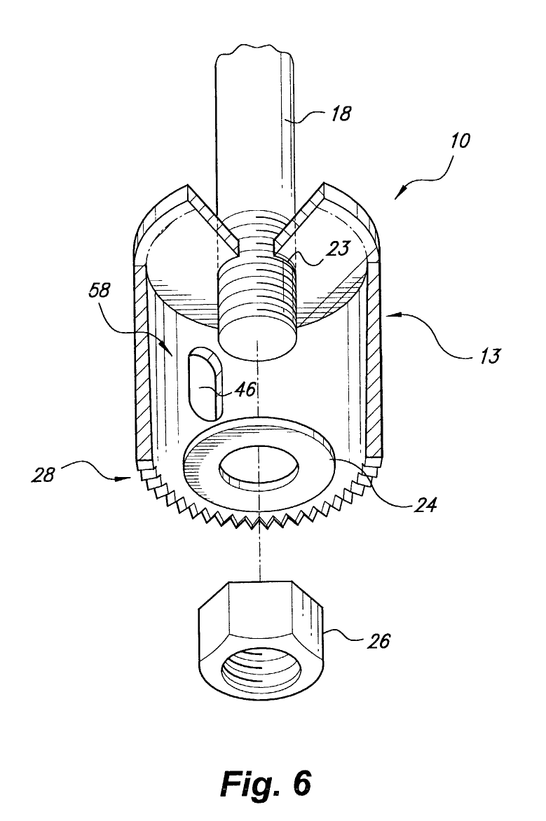

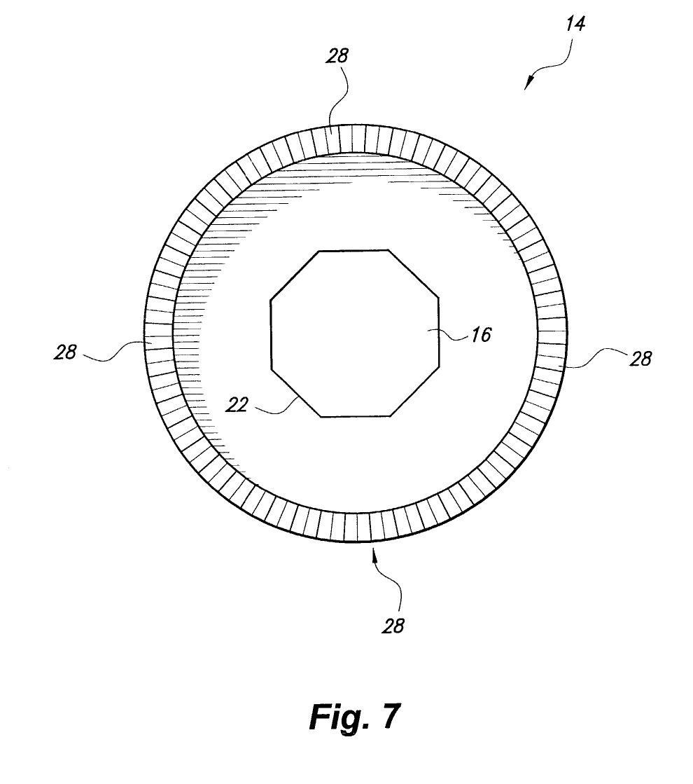

The second end portion 14 of the device 10, illustrated in FIGS. 2, 4, 5A, 5B, 6, 7, 8A, 8B, and 9, is comprised of a plurality of saw-like or serrated cutting surfaces 28 machined into the open-ended edge of the cylindrical body 13 (FIGS. 2 and 6) of the cutting device 10. In a preferred embodiment depicted in FIG. 7, forty-eight equally spaced cutting surfaces 28 are oriented in FIG. 6 with a base 40 (FIG. 8A) of each of the equilateral triangle cutting surfaces 28 (viewed from the side in FIG. 8A) in a plane perpendicular to the longitudinal axis of the cylindrical body 13 as in FIG. 6. The tops 30 (FIG. 8A) of the cutting surfaces 28 point in a direction parallel to the sidewall or axis of the cylindrical body 13. Therefore, the tops 30 and the bases 40 of the cutting surfaces 28 are parallel. The tops 30 of the cutting surfaces 28 in FIG. 8A are separated by a peak P₁ to peak P₂ distance of approximately 0.068 inches. The cutting surfaces 28 have an outside arc r₁ as shown in FIG. 8B of approximately 0.068 inches and an inside arc r₂ approximately 0.060 inches. Each cutting surface 28 has a thickness Th (FIG. 8B) of at least 0.8 mm. The cutting surfaces 28 are substantially shaped in the form of equilateral triangles as shown in the side view in FIG. 8A, wherein each of the three internal angles is 60°.

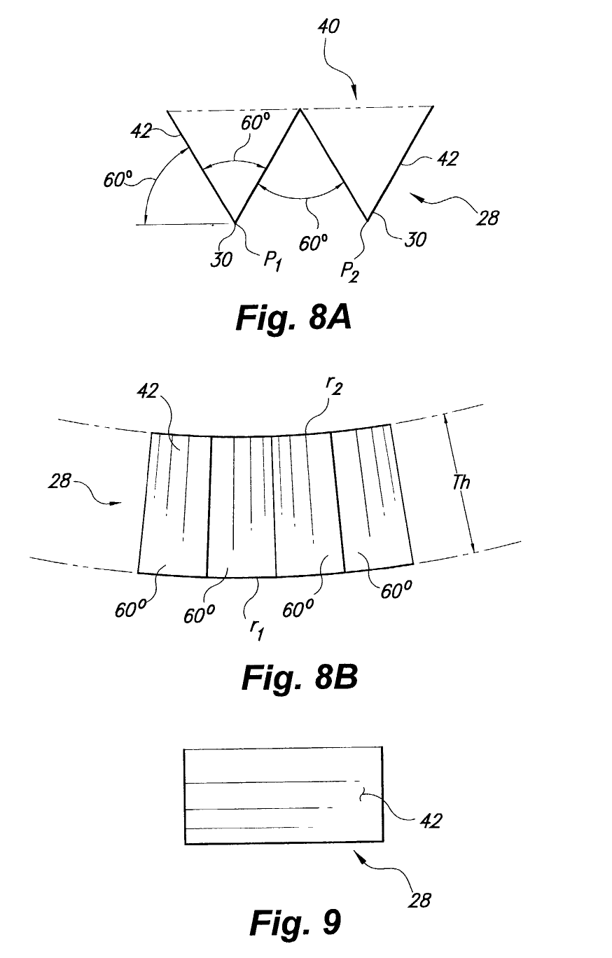

Other conventional saw blades have either non-equilateral triangle or “wolf” shaped teeth. This equilateral triangle configuration of the invention further provides a cutting surface 28 with three equal length sides 42 (FIGS. 8A and 9), which includes the base 40, compared to traditional blades of unequal length sides. The cutting surfaces 28 are neither offset, i.e., leaning inward or outward from the center axis with angled cutting edges on each tooth as in a crosscut saw, nor positioned straight with the sharp edges of the teeth formed at an angle to the planar surface of the blade, as in a ripsaw blade.

FIG. 9 in a front plan view of one inclined side 42 of a cutting edge surface 28 having a rectangular shape. As a result of the equilateral triangle configuration of the cutting surfaces 28, the cutting surfaces can cut in both directions along an arc r₁ (FIG. 8B) when attached to a reciprocal cutting tool or cast cutter machine 20. A known reciprocal or oscillating motion 44 of a slight amplitude, i.e., a back and forth motion 44 is generated by the cutting machine 20 as shown in FIG. 3, thus cutting and holding a cast plug 52 (FIG. 1B). The oscillating motion 44, for example, can have a rotational stroke length of approximately ⅛ inch with a high oscillating speed, for example, 14,000 to 16,000 oscillations per minute. A preferred embodiment of the conventional cast cutter machine 20 is depicted in FIG. 4.

The cutter oscillating motion is generally discussed in U.S. Pat. No. 5,468, 247, “Saw Blade For Powered Medical Handpiece,” issued to Matthai et al. on Nov. 21, 1995, the disclosure of which is incorporated by reference in its entirety. One company, M-PACT of Eudora, Kans. is a manufacturer of such oscillating cast cutters, including the American Orthopaedic Cast Cutter. In contrast, the traditional “wolf” teeth blades are only capable of a unidirectional cutting motion, and cannot be used for a bidirectional cut due to the non-equilateral shape and a high degree of offset.

Alternative embodiments of the above described preferred embodiment of the invention could include variations of the shape of the top 30 of the equilateral triangle shaped cutting surfaces 28, the smoothness of the cutting surface sides 42, the degree of offset for any or all of the cutting surface, various patterns of different shaped cutting surfaces, and/or offset cutting surfaces around the second end portion 14, variations of the spacing between the cutting surfaces 28, and variations of the angle of the base 40 (FIG. 8A) of the cutting surface 28 (FIGS. 8A, 8B and 9) in relation to the length of the elongated tube 13.

Alternate embodiments could include the use of equilateral triangular cutting surfaces 28, wherein the top 30 of the cutting surfaces 28 could be reduced in length through angular cuts or, alternately, the top of the cutting surface having a plurality of notches. Similarly, the sides of the cutting surfaces could be configured to have a rough texture or contain notches instead of a flat surface. Further, at least one cutting surface may need to be offset to optimize a cut in a cast 50. As a result, various patterns of differently shaped cutting edges with different degrees of offset can be used to maximize cast cutting. Finally, the angle between the base 40 (FIG. 8A) of at least one cutting surface 28 and the axis of the cylindrical body 13 can be other than 90°, i.e., a perpendicular relationship.

It is to be understood that the present invention is not limited to the sole embodiment described above, but encompasses any and all embodiments within the scope of the following claims.

I claim:

1. A medical cast aerator hole cutting device comprising:
a) a one-piece cylindrical body having a first planar end portion and an opposed second open end portion connected by a sidewall, and said body having a longitudinal axis;
b) said first end portion being adapted for receiving a driving mandrel; and
c) a plurality of equilateral triangle shaped cutting surfaces integrated into the sidewall at said second end portion;
whereby, a pilot bit is obviated in the coring of a medical cast, and whereby said cutting surfaces are configured for cutting an aerating hole in a cast when rotated in a reciprocating clockwise-counterclockwise motion.

2. The medical cast aerator hole cutting device according to claim 1, wherein said cutting surfaces have sharp tops and bases which are formed in parallel and perpendicular to the longitudinal axis of the body.

3. The medical cast aerator hole cutting device according to claim 1, wherein the sidewall has a thickness increasing in thickness from the first end portion to the second end portion.

4. The medical cast aerator hole cutting device according to claim 1, wherein said cutting surfaces have rectangular faces which are substantially parallel to each other.

5. The medical cast aerator hole cutting device according to claim 1, wherein at least one portal slot is located in the sidewall.

6. The medical cast aerator hole cutting device according to claim 1, wherein two portal slots are located in the sidewall and diametrically opposed.

7. The medical cast aerator hole cutting device according to claim 1, wherein the tops of the cutting surfaces have a reduced length with a plurality of angular cuts.

8. The medical cast aerator hole cutting device according to claim 1, wherein the tops of the cutting surfaces have a reduced length with a plurality of notches.

9. The medical cast aerator hole cutting device according to claim 1, wherein said cutting surfaces have smooth flat surfaces.

10. The medical cast aerator-hole cutting device according to claim 1, wherein said cutting surfaces contain notches.

11. The medical cast aerator hole cutting device according to claim 1, wherein said cutting surfaces have a rough texture.

12. The medical cast aerator hole cutting device according to claim 1, wherein at least one of said cutting surfaces has a degree of offset for optimizing a cut.

13. The medical cast aerator hole cutting device according to claim 1, wherein said cutting surfaces have varying patterns of different degrees of offset.

14. The medical cast aerator hole cutting device according to claim 1, wherein said cutting surfaces have varying patterns of spacing between the cutting surfaces.

15. The medical cast aerator hole cutting device according to claim 1, wherein said cutting surfaces have varying patterns of different shapes.

16. The medical cast aerator hole cutting device according to claim 1, wherein the angle of at least one said cutting surface has a base with the longitudinal axis other than a perpendicular relationship.
